(19) United States
(12) Patent Application Publication
(10) Pub. No.: US 2020/0237306 A1
(43) Pub. Date: Jul. 30, 2020

(54) Earhook Assembly

(71) Applicant: Michael Edward LaBrecque, Las Cruces, NM (US)

(72) Inventor: Michael Edward LaBrecque, Las Cruces, NM (US)

(21) Appl. No.: 16/873,047

(22) Filed: Jan. 21, 2020

Related U.S. Application Data
(60) Provisional application No. 62/918,328, filed on Jan. 28, 2019.

(51) Int. Cl. A61B 5/00

(52) U.S. Cl. CPC A61B 5/6816; A61B 5/02438; A61B 5/6838

(57) ABSTRACT

A earhook assembly for placement along the outside of a person's ear supported by the ear flap and ear lobe into which is placed a sensor for receiving and transmitting human body's analytics such as pulse or blood oxygen saturation.

### PROVISIONAL PRIORITY DATE

This application claims the benefit of an earlier filed provisional application, filed Jan. 28, 2019, Application Ser. No. 62/918,328.

### BACKGROUND OF THE INVENTION

### Field of the Invention

This invention relates to an earhook assembly.

### Description of the Prior Art

Assemblies used to allow placement of devices next to a person's ear such as ear buds for listening to music are prevalent throughout modern culture. In the medical field, these assemblies allow receiving and transmission human body analytics such as pulse rate or blood oxygen saturation. Earhook assemblies can be generally place into two categories, ones that fit within the ear canal and ones that are placed on the ear using the earlobe. US Patent Application 2004/0054291, Inventor Christian Schulze, et al, for Pulse Oximetry Ear Sensor, is an example of an earhook assembly in the medical field wherein the assembly is placed on the outside of the ear along the ear flap and earlobe. US Patent Application 2005/0033131, Inventor Yunqunn Chen, et al, for Ear Sensor Assembly and US Patent Application 2014/0288447, Inventor Michael Edward Smith Luna, et al, for Ear Related Devices Implementing Sensors to Acquire Physiological Characteristics, are examples of assemblies that are placed into the ear canal. What all these examples have in common is that each is designed to house a permanent sensor. If the sensor fails, the earhook assembly is replaced. Additionally, these types of earhook assemblies generally result in an uncomfortable attachment to a person's ear for the reason that the sensors employed require tighter earlobe support resulting in a pinching of the ear lobe or are uncomfortable because they are placed into a person's ear canal.

Clearly it can be appreciated in the art that upon the failure of the sensor, if only the sensor could be replaced and not the entire earhook assembly, such approach would be desired both from a cost as well as a ease of use approach. Additionally, a more comfortable attachment to a person's ear is desired.

### SUMMARY OF THE INVENTION

It is, therefore, an object of the present invention to provide a simple earhook assembly to be used along with a sensor that will receive and transmit human body analytics. In the event that the sensor fails, such sensor can be replaced without the need to replace the entire earhook assembly. Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a side view of the present invention.

FIG. 2 depicts a frontal, rear, top and side view.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

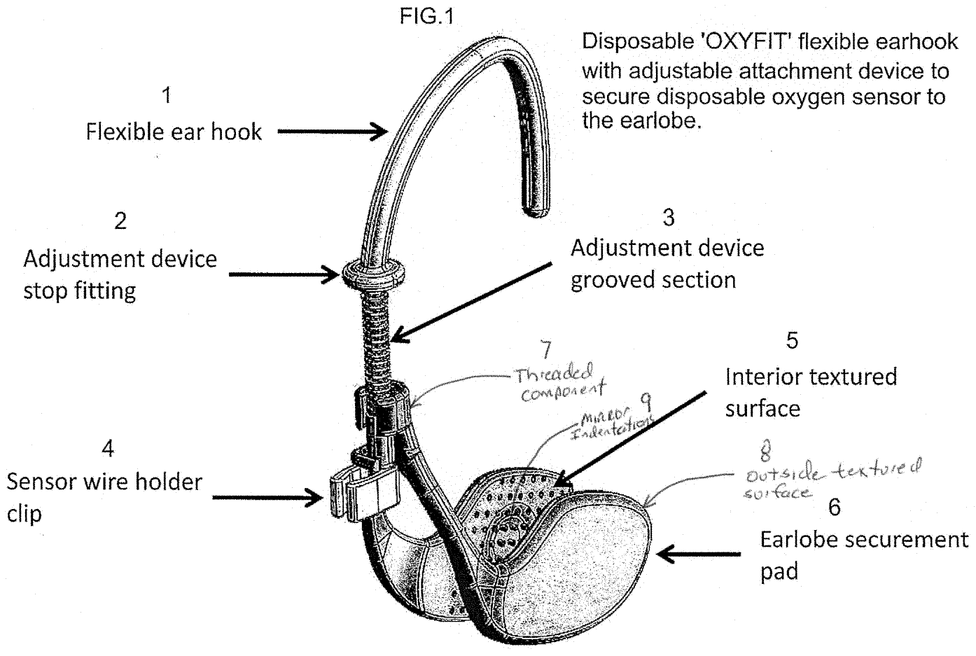

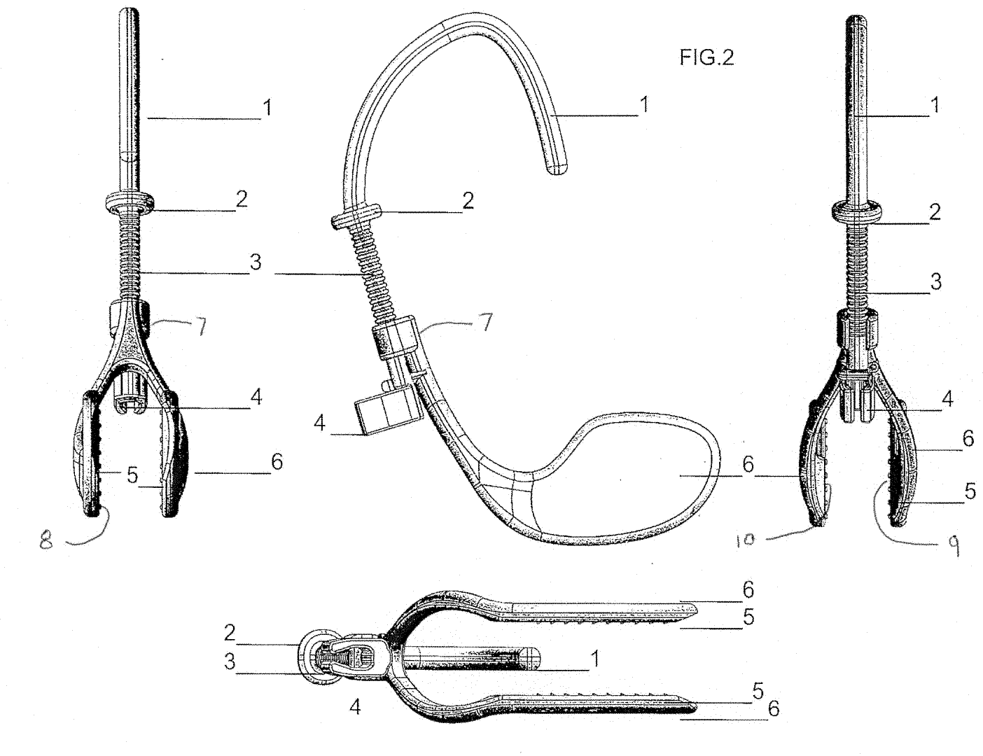

The earhook assembly of the present invention, depicted in both FIGS. 1 and 2, is made from any material of sufficient rigidity and strength to be supported by the ergonomically designed earhook [1] along the top, inside edge of a person's ear flap. The earhook [1] is securely attached to an adjustment device comprising a stop fitting [2], and a grooved section [3]. An earlobe securement pad [6], comprising a surface that will contact and fit upon the inside surface of the earlobe [5] as well as a second surface that will contact and fit securely upon the outside surface of the earlobe [8]; connected together at the threaded component [7] of the grooved section [3] of the ear hook [1]. As shown in FIGS. 1 and 2, the inside surfaces of the earlobe securement pad can be textured to form a more secure attachment. The bottom portions of the inside [5] and outside surfaces [8] are not connected together so that said surfaces can be placed one on each side of a person's earlobe. The top of the inside [5] and outside surfaces [8] are connected together at the threaded component [7] using any convention means such as glue or heat welding. Both the inside [5] and outside surfaces [8] of the earlobe securement pad [6] include identical mirror indentations [9 and 10] to securely accept and hold a sensor for receiving and transmitting human body analytics such as pulse and/or blood oxygen content. Other body analytics can be measured using the sensor such as temperature and blood pressure depending on the type of sensor used. While not necessary for the use of the current invention, FIGS. 1 and 2 both depict a sensor wire holder clip [4] attached at the bottom of the grooved section [3]. The threaded component [7] mates with and rotates along the grooved section [3]. As the threaded component [7] is rotated, the earlobe securement pad [6] will move up or down on the grooved section depending on the direction of the rotation. This movement allows adjustment of the earhook assembly [1] to better fit a person's ear.

While the preferred embodiments have been shown and described, it will be understood that there is no intent to limit the invention by such disclosure, but rather, is intended to cover all modifications and alternate constructions falling within the spirit and scope of the invention as defined in the appended claims.

1. An earhook assembly comprising an earhook ergonomically designed to fit along the top, inside edge of a person's ear flap; and an earlobe securement pad with identical mirror indentations to securely accept and hold a sensor for receiving and transmitting human body analytics.

2. The earhook assembly of claim 1 wherein the top of the inside and outside surfaces of the earlobe securement pad are connected together using any convention means.

3. The earhook assembly of claim 1 further comprising a sensor wire holder clip.

4. An earhook assembly comprising an earhook ergonomically designed to fit along the top, inside edge of a person's ear flap; an adjustment device with stop fitting; a threaded component; a grooved section; and an earlobe securement pad with identical mirror indentations to securely accept and hold a sensor for receiving and transmitting human body analytics.

5. The earhook assembly of claim 4 wherein the threaded component mates with and rotates along the grooved section.

6. The earhook assembly of claim 4 wherein the top of the inside and outside surfaces of the earlobe securement pad are connected together at the threaded component using any convention means.

7. The earhook assembly of claim 4 further comprising a sensor wire holder clip attached at the bottom of the grooved section.
